(19) United States
(12) Patent Application Publication
(10) Pub. No.: US 2018/0338721 A1
(43) Pub. Date: Nov. 29, 2018

(54) A WEARABLE DEVICE FOR MEASURING A PHYSIOLOGICAL PARAMETER OF A USER AND A MEASUREMENT METHOD

(71) Applicant: KONINKLIJKE PHILIPS N.V., Eindhoven (NL)

(72) Inventors: Lu Wang, Eindhoven (NL); Lucas Jacobus Franciscus Geurts, Best (NL); Vincentius Paulus Buil, Veldhoven (NL); Jia Du, Waalre (NL); Cornelis Petrus Hendriks, Eindhoven (NL); Daan Anton van den Ende, Breda (NL); Franciscus Johannes Gerardus Hakkens, Eersel (NL)

(21) Appl. No.: 15/761,544

(22) PCT Filed: Sep. 21, 2016
(86) PCT No.: PCT/EP2016/072337

§ 371 Date: Mar. 20, 2018

(30) Foreign Application Priority Data
Sep. 21, 2015 (EP) 15185988.1

(51) Int. Cl. A61B 5/00; A61B 5/0205

(52) U.S. Cl. CPC A61B 5/6843; A61B 5/6831; A61B 5/0205; A61B 5/7221; A61B 5/7282; A61B 5/021; A61B 5/6824; A61B 5/6828; A61B 5/6829; A61B 5/6823; A61B 5/6814; A61B 5/6826

(57) ABSTRACT

A strap-based wearable device for measuring a physiological parameter of a user. A sensor arrangement is used to convey information about the physiological parameter of the user. The tightness of the strap arrangement is controlled automatically in response to the quality of the sensor signals.

### FIELD OF THE INVENTION

This invention relates to wearable devices for monitoring user signals where the devices have an automatically adjustable strap.

### BACKGROUND OF THE INVENTION

There is an increasing interest in wearable sensor devices. These can be used to provide real time medical information, or to provide physiological performance information for example for use in physical training.

Such wearable devices usually have intimate contact with the user, so that comfort is vital for the acceptance of such devices, especially when such wearable devices are to be worn all day long.

Some measurements require tight physical contact with the user, for example for pulse measurement based on pressure signals, or for electrical conductivity measurement. Indeed, smart watches exist which include such pulse measurement capability. For periodic measurements, the tightness is not needed at all times, and only when measurements are being made.

One of the reasons that people show reluctance to wear devices like a smart watch is that these devices may not feel comfortable (being too tight) to wear for the length of the day.

There is therefore a need for a wearable device for measuring a physiological parameter of a user which can provide user comfort while also being able to extract the desired physiological information in a reliable way.

### SUMMARY OF THE INVENTION

The need is at least partly fulfilled by the invention. The invention is defined by the independent claims. The dependent claims provide advantageous embodiments.

The device as defined by the invention includes a sensor carried by a strap arrangement. The strap arrangement allows that the device can be worn by a user such that the sensor contacts or presses against the skin of the user. The device is able to adjust its strap tightness to maintain or improve the quality of sensor signals received. It also means that when sensor signals are not needed, the strap tightness may be reduced for increase of comfort. These adjustments may be made without requiring physical control by the user. The signal quality may for example be an indication that a signal of suitable amplitude is present, or a required signal to noise ratio is achieved.

The sensor arrangement may for example be for measuring one or more of: heart rate; blood flow rate; blood pressure, blood gas-saturation level and skin conductance. But others are also possible. The heart rate and blood parameters sometimes require a certain pressure to be exerted on skin parts in order to reliably measure the parameters. Similarly, blood gas saturation level measurement, if done optically, may require that environmental light is prohibited from disturbing the actual measurement using a light source. Hence skin contact may need to be established at all, or over a larger area than in sensor rest mode. Thus, by way of example, a heart rate may be monitored optically or based on pressure or vibration sensing. Each of these measurement devices preferably has a predetermined (sometimes) firm contact with the skin and thereby may require a contact pressure with the user which they may not wish to maintain at all times.

Environmental sensors may also be provided, for example a water sensor or an ambient light sensor. Information from such sensors may also be used to determine how tight the strap arrangement needs to be in order to obtain the desired signal quality of the sensor signals.

The strap adjustment system may comprise one or more of:

an electrically driven shape change structure;

a light driven photomechanical structure;

a temperature driven structure; and

a magnetic field driven structure.

These are different possible mechanisms for inducing a change of shape (which then translates to a change in strap tightness) based on a control variable. The control variable is then generated by the device, such as an electric control signal, an optical output, a temperature level achieved by a heater or a magnetic field strength.

The strap adjustment system may instead comprise a chemically induced adjustment mechanism.

The controller may be adapted to assess the quality of the sensor signals based on one or more of:

discontinuation of a sensor signal being received;

presence of an unexpected peak or pattern in the sensor signal; and

presence of high levels of noise in the sensor signal.

These indicators may be used to detect a deterioration in the signal quality.

In one preferred example, the physiological parameter comprises a heart rate. The strap arrangement may for example comprise a wrist strap, to enable heart rate monitoring at the wrist. The device may therefore comprise a smart watch.

The controller may be adapted to implement a measurement mode of operation and a non-measurement mode of operation, wherein the wrist strap is tighter during the measurement mode of operation. The non-measurement mode of operation may for example have the strap much looser, so that the watch can if desired be worn like a loose wrist bracelet.

According to examples in accordance with another aspect of the invention, there is provided method of measuring a physiological parameter of a user using a wearable device which comprises a strap arrangement, a sensor arrangement carried by the strap arrangement for pressing against the skin of the user, for generating sensor signals which convey information about the physiological parameter of the user, and a strap adjustment system for adjusting the tightness of the strap arrangement, wherein the method comprises:

assessing the quality of the sensor signals; and

controlling the strap adjustment system in response to the quality of the sensor signals.

This method ensures that suitable sensor signals are obtained, in an automated manner.

### BRIEF DESCRIPTION OF THE DRAWINGS

Examples of the invention will now be described in detail with reference to the accompanying drawings, in which:

FIG. 1 shows the components of a device for monitoring a physiological parameter;

FIG. 2 shows one example of the device implemented as a smart watch;

FIG. 3 shows a first example of output display to the user to indicate strap adjustments being made;

FIG. 4 shows a second example of output display to the user to indicate strap adjustments being made;

FIG. 5 shows a third example of output display to the user to indicate strap adjustments being made;

FIG. 6 shows a first example of implementation of a strap tightening system; and

FIG. 7 shows a second example of implementation of a strap tightening system.

### DETAILED DESCRIPTION OF THE EMBODIMENTS

The invention provides a strap-based wearable device for measuring a physiological parameter of a user. A sensor arrangement is used to convey information about the physiological parameter of the user. The tightness of the strap arrangement is controlled automatically in response to the quality of the sensor signals. In this way, the device reacts and adjusts itself according to the sensor signals.

Wearable devices that react to motion or activity have been proposed. For example, a smart bra has been proposed, which can stiffen or loosen the bra strap and the breast cup based on strain, (mechanical) stress, breathing and breast movement. The smart bra performs an actuation to improve the comfort level. However, this system does not react to quality of measured signals to ensure accurate sensing information is gathered.

It is also known to monitor sensor signal quality. For example, the continuous heart rate monitor watch known as the MIO Alpha (trade mark) watch monitors heart rate measurement quality and it alerts the user with an audible sound after a long duration of detected poor quality signal. The user may then temporarily fasten the watch more, during exercise. For a prolonged duration, this causes discomfort for the user.

Wearable devices are increasingly being seen as decorative accessories. A decorative wrist band or bracelet is usually worn loosely hanging on a person's wrist. There is a therefore a conflict between the desired aesthetic appearance and the functional requirements, which functional requirements dictate that the device must be positioned correctly and tightly when in a measuring mode.

The invention thus enables the tightness to be adjusted during measurement intervals to ensure signal quality (and avoid excessive discomfort), and during non-measurement intervals to meet aesthetic needs (as well as comfort). Automatic mechanical actuation is provided to adjust the distance to the skin, and/or the contact pressure with the skin, based on the determined measurement quality of the sensor signals when in the measurement mode. In this way, a balance between measurement quality, comfort and aesthetics is provided.

The strap tightness may be controlled using any component or material which can change shape in response to an applied stimulus. Various examples will be presented below. The tightness adjustment is to bring a sensor closer to the skin or to a fixed position to ensure optimal measurement quality. The strap tightness adjustment is controlled using a closed-loop control approach. Tightness may apply when the strap of the device is around a specific body part such as a finger, arm, wrist, leg, ankle, belly or waste, chest, neck, head etc.

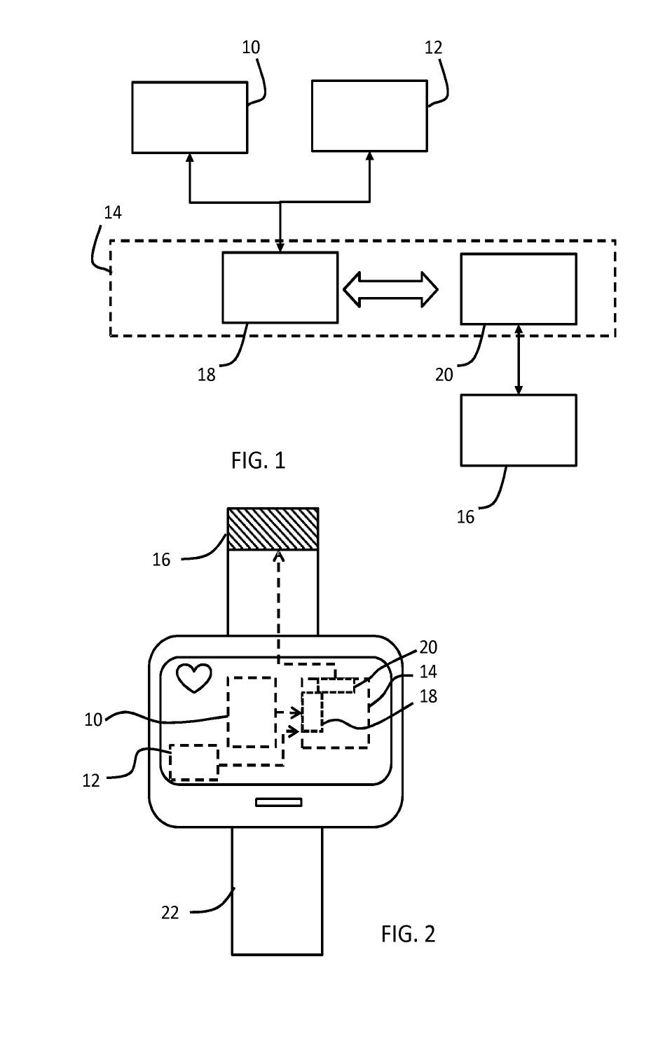

FIG. 1 shows generally the different components of the system.

A sensor arrangement 10 measures the vital signs being monitored which comprise one or physiological parameters of the user. The sensor arrangement is thus for vital sign measurement. The parameters may comprise one or more of the heart rate, blood flow, or skin conductance.

The sensor arrangement 10 is pressed against the skin for generating sensor signals which convey information about the physiological parameter of the user. The sensor arrangement 10 is held in place by a strap arrangement, not shown in FIG. 1.

FIG. 1 also shows optional environmental sensors 12. These may for example detect ambient light and/or water presence. The signals from these sensors are able to communicate whether the measurement is disturbed by the environment so that corrective action may be taken to ensure that the sensor signals correctly convey information about the physiological parameter being monitored.

For example, the ambient light level may alter the way an optical sensor signal is to be interpreted, and the presence of water may alter the way a conductivity sensor signal is to be interpreted.

The system is controlled by a controller 14. The controller 14 assesses the quality of the sensor signals. It can then control a strap adjustment system 16 in response to the quality of the sensor signals. The strap adjustment system comprises actuators for actuating the device strap.

FIG. 1 shows that the controller 14 has to two types of control module; a measurement control module 18 and an actuation control module 20.

The measurement control module 18 receives the sensor signals and interprets them, in particular to assess the signal quality. The device may have a measurement mode and a non-measurement mode. The measurement control module may be used to set the measurement mode intervals and timing. It is also used to communicate the sensor signal quality (such as “normal”, “abnormal”, “worsened” or “improved”). It may also monitor parameters other than the physiological parameter, such as motion status and the environmental sensor signals.

The actuation control module 20 controls the actuator or actuators 16. The two control modules communicate so that the measurement control module 18 knows when a strap adjustment has been completed, and it can instruct the actuation control module 20 to make required adjustments.

The two control modules implement a closed loop control by communicating with each other.

The actuators 16 may take a variety of forms.

Electroactive Polymers (EAPs) may provide physical deformation in response to an electrical stimulus. Electroactive polymers (EAP) are an emerging class of materials within the field of electrically responsive materials. EAPs can work as sensors or actuators and can easily be manufactured into various shapes allowing easy integration into a large variety of systems.

Materials have been developed with characteristics such as actuation stress and strain which have improved significantly over the last ten years. Technology risks have been reduced to acceptable levels for product development so that EAPs are commercially and technically becoming of increasing interest. Advantages of EAPs include low power, small form factor, flexibility, noiseless operation, accuracy, the possibility of high resolution, fast response times, and cyclic actuation.

Devices using electroactive polymers can be subdivided into field-driven and ionic-driven materials.

Examples of field-driven EAPs are dielectric elastomers, electrostrictive polymers (such as PVDF based relaxor polymers or polyurethanes) and liquid crystal elastomers (LCE).

Examples of ionic-driven EAPs are conjugated polymers, carbon nanotube (CNT) polymer composites and Ionic Polymer Metal Composites (IPMC).

Field-driven EAP's are actuated by an electric field through direct electromechanical coupling, while the actuation mechanism for ionic EAP's involves the diffusion of ions. Both classes have multiple family members, each having their own advantages and disadvantages.

The actuators may instead comprise Shape Memory Alloys (SMAs). Shape memory materials (SMMs), especially shape memory alloys (SMAs), are able to provide significant force and stroke when heated beyond their specific phase change temperature. Even if the dimensions of the material are small, the force and stroke delivered are, relative to these dimensions, very high and accurate, over a very long period of time and after many switching operations.

SMAs are thus actuated by causing heating, for example by operating a Peltier heating element or a galvanic wire heater. After the temperature rise and shape change due to the phase change, the material must be brought back to the original shape, before an actuation can be restarted. This may be achieved by forming the material with pre-stress, or using a separate biasing element, so that after cooling, the pre-stress returns the material to the original state.

The pre-stressing is needed because when there is a temperature decrease, the phase changes back to the original phase, but the shape does not. Thus, before the actuator can be used again, after a temperature decrease, an external actuation must be initiated to reverse the shape change of the SMM.

The two main types of shape memory alloys are copper-aluminum-nickel, and nickel-titanium (NiTi), which is known as Nitinol. SMAs can however also be created by alloying zinc, copper, gold and iron. SMMs can exist in two different phases, with three different crystal structures (i.e. twinned martensite, detwinned martensite and austenite).

Although iron-based and copper-based SMAs, such as Fe—Mn—Si, Cu—Zn—Al and Cu—Al—Ni, are commercially available and cheaper than Nitinol, Nitinol based SMAs are more preferable for most applications due to their stability, practicability and superior thermo-mechanic performance.

The actuators may instead comprise Hydrogel thin films. A discussion of such materials is given in the article “Advances in Smart Materials: Stimuli-Responsive Hydrogel Thin Film”, by Evan. M. White et. al., Journal of Polymer Science, Part B: Polymer Physics, 2013, 51, 1084-1099. These materials may be controlled to induce swelling. The article discusses the use of a heat stimulus, a light stimulus, a mechanical stimulus and a chemical stimulus for example pH-responsive gels.

A further example is piezoelectric actuators including piezoelectric motors, which deform in response to an applied electric signal.

A further example is electrical coil motors, such as small stepper motors.

The use of so-called “smart materials” rather than motors offers particular advantages in terms of low power consumption and small physical size.

The actuation can for example be driven by the following smart materials:

(i) Electric-driven: the shape changing is powered by electric signals (e.g. the EAP example above);
(ii) Light-driven: the shape changing is powered by certain light frequencies of outdoor light, by using photomechanical materials;
(iii) Temperature-driven: the shape changing is energized by temperature (e.g., the SMA example above, and also temperature-responsive polymers);
(iv) Magnetic-driven: the shape changing is powered by the magnetic field (e.g., magnetic shape memory);
(v) Cross linked: the shape changing is activated by a range of triggers such as pH, temperature (e.g. the hydrogel example above).

The devices all have in common that they rely on a material which changes shape in response to a stimulus, and thereby avoid the need for expensive motors or other complex mechanical structures.

As explained above, the control of the actuator may take place in a measurement mode. This mode may be set by the user. For example, when going for a run, and wanting heart rate or other physiological information to be gathered, the user can specify this. However, the mode selection may be automatic, for example some of the materials listed above could also respond directly to changing environmental conditions. For example going outdoors could trigger light-driven actuation. The actuation is then not (only) electrically controlled but may be controlled or partially controlled by environmental factors.

In other examples, there could be fixed scheduled times for the measurement mode, for example to create datasets that can be compared over hours or days. For example, the heart rate or heart rate variability of a person may be measured at a particular time each morning. An extension of this could be that the measurement is initiated after an external event. For example, a measurement may be made automatically a fixed time after the person has woken up. This may be detected based on movement of the wristwatch incorporating the sensor, or else based on an external alarm which is assumed to indicate the time when the person wakes up.

The actuation signal for example comprises one of three control signals: loosen, tighten and no change. A loosen command is made when the non-measurement mode starts or when previous over-tightening is indicated. Conversely, a tighten command is made when a measurement mode is in place, and signal deterioration is detected, or else an environmental disturbance is sensed. No change is needed if the measurement quality is stable during the measurement mode.

FIG. 2 shows the device of FIG. 1 implemented as a smart watch. The same components are given the same reference numbers. The strap is shown as 22.

Mechanisms for implemented automated tightening of a wrist watch strap are known. For example U.S. Pat. No. 8,370,998 discloses a watch strap which includes a torsion spring which generates a torsion force which rotates a pin to tighten the strap. Instead of using a torsion spring (which is not controllable by an external input), one of the actuators described above may be used to induce tightening of the strap.

The watch may provide heart rate and activity monitoring, and smart watches with this functionality are already known. Thus, the required physiological sensors are already well-known and available. The heart rate monitoring may use optical or pressure sensing. A chest worn device may have ECG electrodes and microphones.

Ambient light detection is also known in wearable devices. For example the MIO Alpha watch referenced above also uses an optical sensor to detect ambient light.

Various commercial rain sensors also exist, such as a rain drop detection sensor from the company Hydreon.

The measurement control module is used to assess the quality of sensor signals once a measurement mode is in place. Some devices may be for permanent patient monitoring for example, in which there is no non-measurement mode and the device is actively monitoring the sensor signals all the time.

The assessment to be made will be based on the type of measurement, such as heart rate, blood flow, or skin conductance. Abnormal signals, such as discontinued signals, unexpected peaks or patterns or excessive signal disturbance, are communicated to the actuation control module.

Algorithms that assess heart rate measurement quality are well-known. For example, reference is made to the article by C. Yu, Z. Liu, T. McKenna, A. T. Reisner, and J. Reifman, “A Method for Automatic Identification of Reliable Heart Rates Calculated from ECG and PPG Waveforms,” J Am Med Inform Assoc, vol. 13, no. 3, pp. 309-320, 2006.

In the simple version of FIG. 2, a single actuator may be used to control the strap tightness. However, a more complicated device may have multiple actuators for the single strap or else multiple straps, so that there are more degrees of freedom in the control of the device. In such a case, different parts of the wearable device may be controlled independently, and for this purpose, the location of those sensors which receive reduced quality sensing signals may also be communicated to the actuation control module 20.

Following the actuation function, the measurement control module re-assesses the physiological signals to verify the effectiveness of the actuation. If the actuation tightens the strap excessively, which results in restricted blood flow, it may for example be detected by observing reduced peaks in the signal. Consequently, there may be an iterative process of tightening and loosening as part of the closed loop control.

For a system which implements periodic measurement modes, once a measurement interval ends, the measurement control module may then send a ‘loosen all actuators’ signal to the actuation control module. The device may also have an override function, by which the user can input an interruption signal.

The device has a display to convey information to the user. This may include information about the strap tightness setting or adjustments being made.

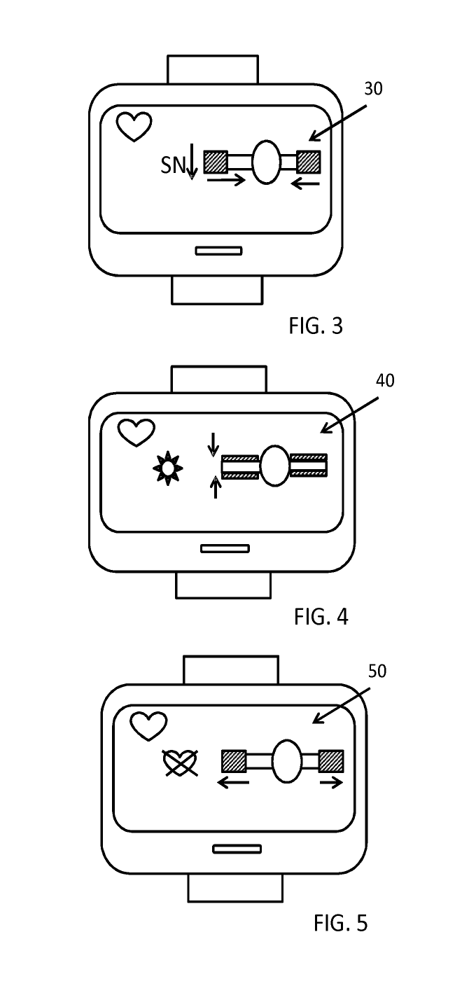

FIG. 3 shows an example of a smart watch display, when a reduction in signal to noise ratio is detected during a measurement mode. The signal to noise reduction is conveyed to the user as well as a symbol 30 indicating that strap tightening is being conducted.

This reduction in signal quality may for example arise after a user starts running, and it may for example be a heart rate measurement which has reduced signal quality. After tightening, a re-assessment of measurement quality is repeatedly conducted. After a further time, worsened heart rate measurement quality may again be detected, so that the watch strap tightens further.

FIG. 4 shows a strap tightening necessitated by environmental disturbance. The user may for example be taking a walk on a very sunny day. An ambient light sensor will then detect light disturbance.

FIG. 4 relates to a strap design with different actuation functions. The adjustment which needs to be made may be to block ambient light rather than tightening the strap. This may involve curling the strap over to shield a light sensor from the sides. This is represented by the symbol 40.

FIG. 5 shows a symbol 50 indicate a requested interruption by the user. The user may not be satisfied with the comfort level, so has the option to pull (or hold) the strap to interrupt the measurement. When this interruption is sensed, the device concludes that user interruption is required. It stops measurement and loosens the strap.

Some examples will now be given to show how the strap tightening arrangement may be implemented.

FIG. 6 shows an example based on a shape memory alloy (SMA). The strap 22 includes SMA wire actuators 60 embedded in the electrically insulating flexible strap 22. The actuators are heated by direct current driving, for example to a maximum temperature of around 70 degrees.

The strap further includes springs 62 which are used to provide a detwinning force to return the strap to its original shape after cooling. In this way a reversible actuation is enabled. The required spring function may instead be generated by the material of an elastic strap without requiring additional returning springs.

The image on the left shows the strap in plan view. The arrows 64 show the change in length induced by heating.

The image on the right shows the strap tightening effect as arrow 66.

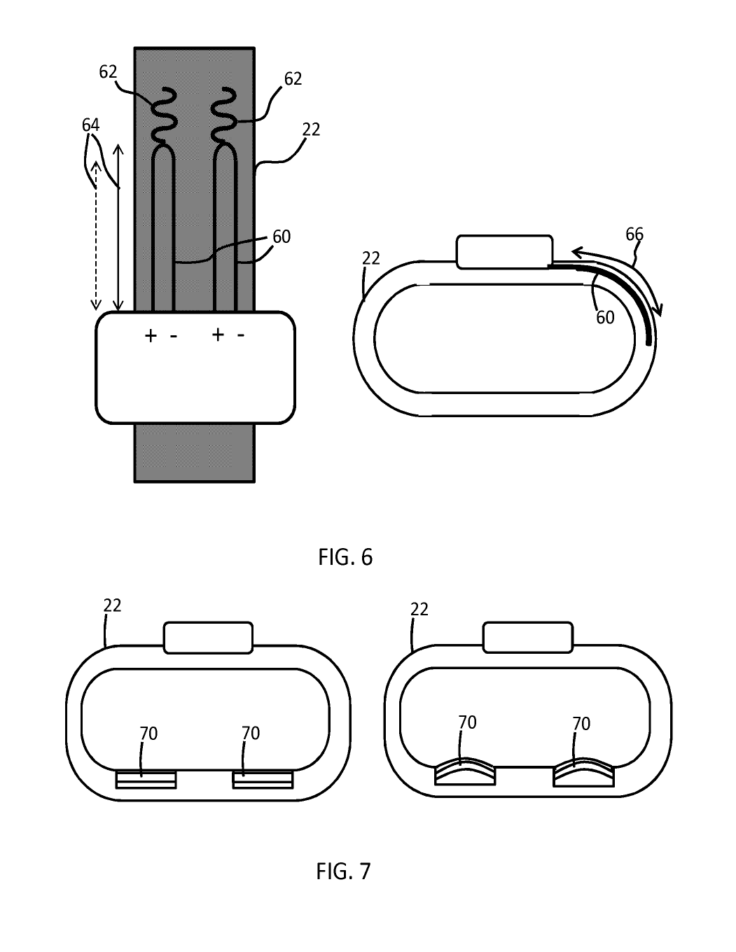

FIG. 7 shows an example based on an electroactive polymer actuator. Two bending actuators 70 are shown, which extend across the width direction of the strap. The actuators may be positioned at any location around the strap 22.

FIG. 7 shows the non-actuated shape in the left image and the actuated shape in the right image. The bending induced causes a restriction to the size of the opening defined by the strap.

This invention is suitable for smart wearable devices such as watches, wrist bands, waist line straps, as well as smart decorative wearable devices.

Thus, the invention is not limited to a wrist band or smart watch implementation, and it may be used around the waist or chest or indeed other parts of the body.

In the examples above, main signal quality indications in the form of signal amplitudes or a signal to noise ratio have been mentioned. Other indicators for the quality of the sensor data, which may be used as alternatives or in conjunction, include:

discontinuation of a sensor signal being received, such as signal drop outs or no signal received;

presence of an unexpected peak or pattern in the sensor signal;

deviations from an expected signal pattern (such as a beat pattern detected by a beat detector);

large fluctuations in signal strength;

number of signal artifacts within a certain timeframe;

deviations from an expect spectral content based on a spectral measurement.

Examples of possible sensor measurement have been given above of heart rate, blood flow rate and skin conductance.

Other examples include:

heart rate variability;

SPO2;

temperature;

an ECG signal;

respiratory rate;

ultrasound signals using a microphone;

blood pressure;

bio impedance measurements;

blood flow measurements;

body (part) movement and orientation measurements;

muscle tension measurements (using electromyography (EMG));

electrical activity along the scalp (using an electroencephalogram (EEG)).

Examples have been given above of an electrically driven actuator (using an EAP material) and temperature driven actuator (using a SMM). Electrical and thermal actuation are the most suitable options. However, light actuated materials also exist, for example UV actuated materials.

Optically responsive materials are for example based on azo compounds. Mixtures of reactive liquid crystals and reactive azo compounds may form a liquid crystalline state to obtain films with aligned molecules. If this alignment is implemented over large surfaces, a so called mono-domain material is obtained. If small domains are obtained it is called a multi-domain material. Alternatively, polyimides and polyesters exist that are not liquid crystalline but give rise to a similar effect when irradiated. However, the materials have high glass temperatures and the response is therefore very slow.

The response of the LC based responsive materials is driven by the fact that upon E-Z isomerization the order in the polymerized material is decreased leading to a contraction of the material in the direction of the alignment (and at the same time expansion in the other two directions).

An example of an optically responsive actuator is described in WO 2007/086487.

UV irradiation of mono-domain films may be used to give contraction using unpolarized light because the aligned azobenzene groups induce a strong anisotropy in absorption, the absorption parallel to the molecular axis being the highest. If multi-domain films are used, irradiation may be performed with linearly polarized light parallel to the direction of contraction. For the best response, the use of mono domain films is preferred. In order to avoid strange contraction effects due to the expanding in the direction perpendicular to the molecular alignment, it is advisable to use small films with alignment in the length of the film.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

1. A wearable device for measuring one or more physiological parameters of a user, comprising:
a sensor arrangement for contacting the skin of the user and for generating sensor signals which convey information about the one or more physiological parameters;
one or more environmental sensors configured for measuring one or more environmental factors indicating changes of environmental conditions;
a strap arrangement carrying the sensor arrangement;
a strap adjustment system for adjusting the tightness of the strap arrangement; and
a controller,
wherein the controller is for:
assessing the quality of the sensor signals; and
controlling the strap adjustment system in response to the quality of the sensor signals,
wherein the controller is adapted to implement (i) a measurement mode of operation and (ii) a non-measurement mode of operation, wherein the strap is tighter during the measurement mode of operation, and
wherein the controller is configured to automatically select a mode of operation from (i) the measurement mode of operation and (ii) the non-measurement mode of operation in response to environmental factors measured by the one or more environmental sensors.

2. A device as claimed in claim 1, wherein the physiological parameter comprises one of more of: heart rate, blood flow rate, blood pressure, blood gas-saturation and skin conductance.

3. A device as claimed in claim 1, wherein the one or more environmental sensors comprises one or more of: a water sensor and an ambient light sensor.

4. A device as claimed in claim 1, wherein the strap adjustment system comprises one or more of:
an electrically driven shape change structure;
a light driven photomechanical structure;
a temperature driven structure; and
a magnetic field driven structure.

5. A device as claimed in claim 1, wherein the strap adjustment system comprises a chemically induced adjustment mechanism.

6. A device as claimed in claim 1 wherein in the controller is adapted to assess the quality of the sensor signals based on one or more of:
discontinuation of a sensor signal being received;
presence of an unexpected peak or pattern in the sensor signal;
presence of high levels of noise in the sensor signal;

7. A device as claimed in claim 1 wherein the adjustment mechanism comprises an electrically driven actuator for adjusting the tightness of the strap, the actuator comprising an electroactive polymer for providing its actuation.

8. A device as claimed in claim 7, wherein the strap arrangement comprises any one of: a finger strap, an arm strap, a wrist strap, a leg strap, an ankle strap, a waste or belly strap, a breast strap or a head strap.

9. A method of measuring a physiological parameter of a user using a wearable device which comprises: a sensor arrangement for contacting against the skin of the user and for generating sensor signals which convey information about the physiological parameter of the user, one or more environmental sensors for measuring one or more environmental factors indicating changes of environmental conditions, a strap arrangement carrying the sensor arrangement and a strap adjustment system for adjusting the tightness of the strap arrangement, wherein the method comprises the steps of:
measuring one or more sensor signals conveying information about the physiological parameter of the user;
assessing the quality of the sensor signals;
controlling the strap adjustment system in response to the quality of the sensor signals; wherein the method further comprises the steps of:
measuring one or more environmental factors indicating changes of environmental conditions;
automatically selecting a mode of operation of the device from (i) the measurement mode of operation and (ii) the non-measurement mode of operation in response to environmental factors measured by the one or more environmental sensors, wherein the strap is tighter during the measurement mode of operation.

10. A method as claimed in claim 9, wherein the physiological parameter comprises one or more of: heart rate; blood flow rate; blood pressure, blood gas-saturation and skin conductance.

11. A method as claimed in claim 10, further comprising sensing the presence of water and/or the level of ambient light, for use in assessing the quality of the sensor signals.

12. A method as claimed in claim 9 comprising assessing the quality of the sensor signals based on one or more of:
discontinuation of a sensor signal being received;
presence of an unexpected peak or pattern in the sensor signal;
presence of high levels of noise in the sensor signal;

13. A method as claimed in claim 9, comprising monitoring a heart rate using a wrist mounted device.
